(19) United States
(12) Patent Application Publication
(10) Pub. No.: US 2010/0094168 A1
(43) Pub. Date: Apr. 15, 2010

(54) INSTRUMENT FOR THE MEDICAL EXAMINATION OF TIGHT BODY DUCTS

(76) Inventors: Siegfried Riek, Rottweil (DE); Karl-Heinz Bachmann, Villingendorf (DE); Thomas Gaiselmann, Villingendorf (DE); Diethelm Wallwiener, Tubingen (DE)

Correspondence Address: THE NATH LAW GROUP, 112 South West Street, Alexandria, VA, 22314, US

(21) Appl. No.: 12/450,330

(22) PCT Filed: Mar. 13, 2008
(86) PCT No.: PCT/EP2008/002002

§ 371 Date: Oct. 6, 2009

(30) Foreign Application Priority Data
Mar. 23, 2007 (DE) 10 2007 014 634.7

(51) Int. Cl. A61B 10/02

(52) U.S. Cl. 600/567

(57) ABSTRACT

The invention relates to an instrument for the medical examination of tight body ducts, particularly for the mammary ducts of the breast, comprising a cannula having a cannula shaft (6) and a distal cannula end (1). The distal cannula end (1) is transparent and has a point (la) that is distally arched, and a tube section (lb) connected thereto. A recess (4) is configured in the tube section (lb). An adenoid curette (2) may be axially displaced on the tube section (lb) in order to separate tissue accommodated in the recess (4). An endoscopic optical element (3) may be inserted into the cannula shaft (6), thus enabling the observation of the distal point (la) and the tube section (lb) having the recess (4) from the interior as a wide angle optical element.

The invention relates to an instrument for the medical examination of narrow ducts in the body according to the general terms of patent claim 1.

During ductoscopy, lenses are inserted in the duct for the examination of narrow ducts in the body. For further diagnosis, tissue samples can also be removed from the body ducts by means of biopsy. Moreover, an important application area is the sample excision of tissue in the milk duct (Ductus lactiferi) of the female breast for the diagnosis of changes in the milk duct, such as adenomas, the preliminary stages of carcinoma, and breast cancer.

Due to the small diameter of the milk duct with a maximum of about 1.2 mm, for a long time it has been possible only to insert an instrument for the sample excision blindly into the duct and to position the instrument relative to the tissue area assigned for the biopsy from the outside by X-ray or ultrasound. Through the miniaturization of the medical endoscope, the possibility has since then arisen of performing a sample excision visually in narrow ducts of the body as well, such as the milk duct of the breast.

From the German patent DE 102 58 483 B3, an instrument is known for this which exhibits a cannula with an outside diameter so small that this cannula can be inserted into the milk duct. In the cannula is disposed an endoscopic lens with a diameter of approximately 1 mm. By means of this lens, through the open, or through a transparent window in the closed, distal tip of the cannula, the area of the duct can be observed in front of the distal tip of the cannula. The cannula exhibits laterally behind the tip a breach in the wall, into which a tissue sample is sucked or is pressed by means of the tissue tonus. The edge of the wall breach is formed as a cutting edge and by moving the endoscopic lens, the tissue sample which has penetrated into the cannula internals through the breach in the wall is pressed against this edge and is cut off. With this instrument, the view is thus made difficult by the endoscopic lens, due to the fact that the body tissue lies on the flat, distal front surface of the lens when moving the cannula and the lens or when the window distally closes the cannula under axial pressure. By means of this pressure, the tissue becomes anemic and appears amorphous, so that a sophisticated examination of the tissue is made difficult. Also, body fluids collecting in front of the lens or the distal cannula window block the view. Finally, with this instrument, the field of view of the endoscopic lens is limited to the distal area lying in front of the tip. The forward movement of the tip of the instrument through the duct is difficult to estimate and consequently orientation in the duct is restricted. Positioning of the breach in the wall can still only be estimated in the area of the tissue to be removed and does not occur in immediate view. The actual process of tissue removal cannot be done visually because the lens here is pressed against the tissue and is covered with blood.

From the German patent DE 100 35 878 A1, an instrument for biopsy is known which consists of a miniature grip instrument with a coaxial lens. Also, with this instrument, the disadvantage cited occurs during insertion of a flat, distal front surface of the lens into the duct. For tissue removal, the view of the lens is completely covered by the biopsate and blood.

The invention is based on the problem of improving an instrument for the examination of narrow ducts in the body so that an inspection and, if need be, a sample excision is possible with a good view.

This problem is resolved according to the invention by an instrument with the features of patent claim 1.

Advantageous embodiments and further details of the invention are given in the subclaims.

With the instrument according to the invention for the examination of narrow ducts in the body, in particular the milk duct of the breast, a distal curved tip is disposed on the distal end of a cannula, which is constructed as transparent. At this curved distal tip, a lateral recess in the outer peripheral surface is enclosed in the cylindrical wall of the cannula. Also, in the area of this recess the distal end of the cannula is constructed as transparent. An endoscopic lens is inserted into the cannula, in which the axial position of the distal end of the lens and its optical properties are chosen so that the field of vision of this lens as well as the curved tip encompass the cylindrical area connecting with the recess.

Upon inserting the instrument into a duct of the body, for example into a milk duct, the curved tip spreads the duct apart, so that the body tissue slides laterally along the tip being moved forward, without an axial pressure of the tissue being able to build up in front of the tip. Also, body fluid contained in the duct flows laterally past to the curved tip. By means of the endoscopic lens, the tissue can be observed to be almost pressure-free sliding past to the tip and the cylindrical, transparent, adjoining area of the cannula, so that the tissue structure is very visible and distinguishable. The feed movement of the instrument and its distal tip in the duct can thus be assessed well. The distal end of the cannula can in this way by careful observation of the tissue lying next to the tip and the adjoining transparent area be positioned so that a conspicuous tissue area lies next to the lateral recess and enters it. Upon entry into the recess, the tissue can relax practically pressure-free, so that it can be inspected without ischemic discoloration or bruising and assessed. Because the recess is closed against the cannula internals, no body fluid or blood reaches into the inside diameter of the cannula in front of the lens, whereby the view could be impeded.

In a preferred embodiment of the instrument, in the outer periphery of the cannula, a knife can be additionally disposed, which can be moved back at the cannula guided over the recess. For a biopsy, the knife can be operated to cut off the tissue found in the recess. The tissue cut off is moreover held in this recess by the knife which has passed over the recess, so that it can be removed by extracting the instrument for a subsequent examination. The cutting off of the tissue can at the same time be observed through the lens. Also, the biopsate cut off and which entered the recess can be observed without blood damaging the lens.

Especially good observation results for the tissue surrounding the distal tip if the distal tip is constructed as a hollow curved dome whose central axis coincides with the central axis of the cannula.

The knife is movable axially or rotationally at the cannula and in an advantageous embodiment is constructed as an adenotome, which encloses the cannula coaxially and is movable axially at the cannula. This embodiment makes the operation of the knife especially simple by means of an operating grip disposed at the proximal end of the instrument.

The adenotome is, moreover, preferably constructed with a distal cutting edge and can be moved between a proximal and a distal end position, in which in the proximal end position, the cutting edge of the adenotome is located proximally behind the recess and is moved in the distal direction back over the recess into the distal end position in front of the recess. At the same time, a radial circular shoulder can serve as a stop for the adenotome.

Because the recess is built into the wall of the distal end of the cannula, the recess presents only a relatively small radial depth. In order to obtain a sufficient amount of tissue material, the recess is suitably constructed as a groove running axially in order to increase the intake capacity of the recess.

The recess can be manufactured in various ways. It is essential that the recess be formed in the outer shell of the cannula end, i.e., it is open outward and is closed against the inside diameter of the cannula.

The recess can be incorporated into the wall of the cannula end, in particular, being cut out, for example, by milling. The wall can also be pressed by deformation radially into the cannula internals in order to form the recess. Finally, it would also be possible to introduce a breach in the wall and into this breach to insert and to attach, to weld, and so forth a container which then forms the recess.

In a further development of the invention, a coagulation device is attached to the outer surface of the knife or of the cannula end, which preferably is constructed as an electrically heatable coagulation zone. If a tissue sample is cut off by the knife, then the coagulation device can be mounted in the area of the incision to coagulate the vessels damaged by the detachment of the tissue sample. The coagulation device can be disposed on the knife so that in cutting off the tissue in the area of the incision, it then also happens that the recess is closed by the knife acting as a cover. This makes additional positioning of the coagulation device unnecessary. In another embodiment, the coagulation device is disposed in an axial or rotational direction and in a definite and thus deliberately traceable manner is moved opposite the recess. Thus, the heat of the coagulation device can be prevented from damaging the tissue sample enclosed and received in the recess.

The cannula shaft can, depending on the application purpose of the instrument, be constructed as a rigid shaft, for example, in the form of a metal tube, or as an elastically flexible shaft, for example, in the form of a plastic tube. In constructing the cannula shaft as a metal tube, the transparent distal end, which envelops the tip and the cylinder area with the recess, preferably made of a transparent plastic, is in particular made as one piece and is fastened to the cannula shaft. If the cannula shaft is constructed as a plastic tube, then the distal end with the tip and the recess is constructed similarly, and they are fastened to this plastic tube. Likewise, it is possible to make the plastic tube of the cannula shaft and the distal end as a single piece out of transparent plastic.

Besides, a flushing channel is additionally disposed in the cannula shaft. Should it be necessary, a suction channel can even be disposed or constructed on the cannula shaft, which leads into the recess at the distal end of the cannula. By means of an underpressure in the suction channel, the tissues to be removed are additionally sucked into the recess. These flushing and suction channels could also be constructed in the space between the cannula shaft and the knife.

In a further development of the invention, it is possible, at the distal end of the cannula, to slide on a marker ring. This marker ring can be stripped off from the distal end of the cannula in the duct at the site at which the sample excision is made, so that it remains at that position in the duct to mark the place. The marker ring is moreover made of a material which is discernible by X-ray or ultrasound. By means of such a marker ring deposited at the site of the sample excision, a tumor which might be diagnosed during histological examination of the tissue removed can easily be found in a subsequent operational intervention. The distal feed movement can be used for stripping off the marker ring, if necessary.

In the following, the invention is clarified in detail using one of the embodiment examples represented in the drawing. It shows:

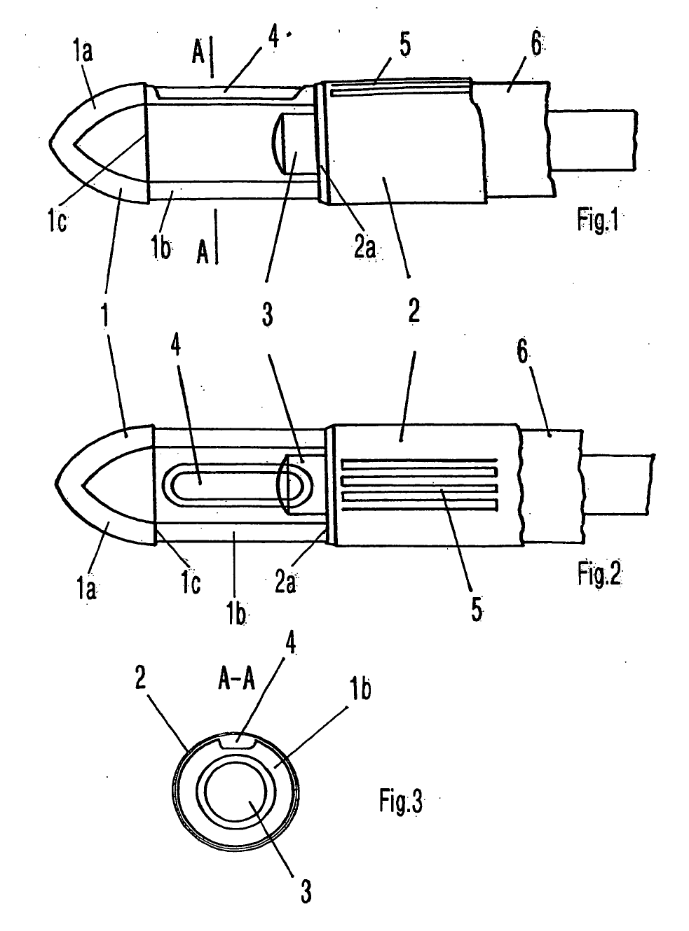

FIG. 1 a side view of the distal end of the instrument,

FIG. 2 a view turned 90° to that of FIG. 1, and

FIG. 3 a cross-section according to the section line A-A in FIG. 1.

The embodiment example represented in the drawing shows an instrument for ductoscopy and in particular for sample excision in the milk duct of the female breast.

Only the distal end of the instrument is depicted in the drawing. The parts of the instrument not depicted corresponds to conventional prior art, so that these do not have to be drawn or described in detail.

The instrument exhibits a cannula with a cannula shaft 6, on the proximal end of which a grip, not depicted, is attached. The cannula shaft 6 can be constructed as a rigid tube, in particular as a metal tube, or as an elastically flexible tube, especially a plastic tube. The distal end 1 of the cannula attached to the cannula shaft 6 is manufactured preferably in one piece.

The distal end 1 consists of a distal tip 1a and a cylindrical tube section 1b attached to it, which attaches to the cannula shaft 6 and with which the cannula end 1 moves over into the cannula shaft 6. The distal tip 1a is constructed as a hollow dome curved in the distal direction, and in particular has a tip 1a in the form of a conical, bulbous curved dome, which is rotationally symmetric about its central axis, in which the central axis of the tip 1a coincides with the central axis of the cannula shaft 6. The tip 1a moves over into the tube section. The tube section 1b is constructed as a hollow circular cylinder, whose outside diameter matches the outside diameter of the cannula shaft 6. The outside diameter of the distal tip 1a is, at the transition to the tube section 1b, somewhat larger than the outside diameter of the tube section 1b, so that a radial circular shoulder 1c is thereby formed at the outside circumference of the cannula end 1.

In the outer shell of the tube section 1b, a recess 4 is constructed, which exhibits the shape of a groove running axially. The recess 4 extends essentially over the entire axial length of the tube section 1b. The radial depth of the recess 4 corresponds almost exactly to the wall thickness of the tube section 1b.

A knife 2 is disposed on the cannula shaft 6. The knife 2 in the embodiment depicted is an adenotome, which encloses the cannula shaft 6 coaxially in the form of a casing. The knife 2 is axially movable on the cannula shaft 6, for which the knife 2 is moved by means of an operating grip, not depicted, disposed at the proximal end of the instrument. The distal peripheral edge of the knife 2 is constructed as a cutting edge 2a. The knife 2 is movable by means of the operating grip between a proximal end position shown in FIGS. 1 and 2 and a distal end position. In the proximal end position, the cutting edge 2a is located proximally behind the recess 4. In the distal end position, the knife 2 is pushed so far forward that the cutting edge\ 2a lies distally in front of the recess 4 and stops at the circular shoulder 1c. In the distal end position, the casing of the knife 2 completely covers the recess.

At the outer surface of the casing of the knife 2 is disposed a coagulation device, which is constructed as an electrically heatable zone 5. The heatable zone 5 can, for example, be achieved with a heating filament introduced at the casing of the knife 2. The surface of the heatable zone 5 is arranged and sized so that it coincides with the recess 4 when the knife 2 is pushed into the distal end position and it covers it completely.

Inserted in the cannula shaft 6 is an endoscopic lens 3. The lens 3 terminates distally in the area of the proximal end of the tube section 1b, i.e. at the proximal end of the recess 4. The lens 3 is constructed as a wide-angle lens, so that the entire inner surface of the cannula end 1, the hollow, curved distal tip 1a, and the tube section 1b with the recess can be observed through the lens 3.

The manner of operation for the instrument is as follows:

The instrument is inserted into a narrow duct in the body, for example a milk duct. Here, the knife 2 is in the proximal end position, so that the entire distal cannula end 1 including the recess 4 are clear off the knife 2. Upon insertion of the instrument, the transparent distal cannula end 1 is pushed into the duct, whereby the tissue of the duct wall slides along the widening distal tip 1a and the attached cylindrical tube section 1b and can be precisely observed through the lens 3. If an interesting tissue area, for example, a tissue area suspected to have a tumor, is visible through the transparent cannula end 1, the cannula end 1 in view through the lens 3 is positioned so that the recess 4 is located at this tissue area. The tissue is at the same time pressed into the recess 4 by its own tonus. If need be, this can be assisted by an underpressure. Now the tissue lying in the recess can be inspected and assessed practically pressure-free. If, moreover, it is known that the tissue located in the recess 4 has to be studied more closely, then the knife 2 is operated and pushed forward distally over the tube section 1b and the recess 4. At the same time, the cutting edge 2a of the knife 2 cuts off the tissue located in the recess 4, which, if necessary, is still aided by stopping the cutting edge 2a at the circular shoulder. The tissue cut off is now enclosed in the recess 4 by the circular casing of the knife 2. The heatable zone 5 can now be brought to the tissue area where the tissue sample was cut off. Through the action of electrical heating, the wound caused by cutting off the tissue sample can be coagulated, especially in order to prevent hemorrhaging. Finally, the instrument is extracted and the tissue sample enclosed in the recess 4 can be withdrawn for histological examination.

### REFERENCE LIST

1 Distal cannula end
1a Distal tip
1b Tube section
1c Circular shoulder
2 Knife
2a Cutting edge
3 Endoscopic lens
4 Recess
5 Coagulation zone
6 Cannula shaft

1. An instrument for the medical examination of narrow ducts in the body, in particular of the milk duct of the breast, with a cannula, which exhibits a cannula shaft and a transparent distal tip, with at least one entrance disposed laterally in the cannula behind the distal tip for a tissue sample and with an insertable endoscopic lens in the cannula,
characterized by the fact that the lateral entrance is constructed as at least one recess (4) in the outer shell of the cannula, that the distal tip (1a) of the cannula is curved in the distal direction, and that the distal cannula end (1) of the tip is transparent up to and at least including the lateral recess (4), so that the field of vision of the inserted lens (3) covers the distal cannula end (1) including the recess (4).

2. An instrument according to claim 1,
characterized by the fact that the distal tip (1a) of the cannula end (1) is constructed as a hollow dome curved in the distal direction, the central axis of which coincides with the central axis of the cannula shaft (6).

3. An instrument according to claim 1,
characterized by the fact that a knife (2) guided at the cannula is movable over the recess (4).

4. An instrument according to claim 3,
characterized by the fact that the knife (2) surrounds the cannula coaxially and is movable axially or rotationally in the circumferential direction at a cylindrical tube section (1b) of the cannula end (1), in which the recess (4) is constructed.

5. An instrument according to claim 1, characterized by the fact that at least one recess (4) is constructed as a cut recess worked into the outer shell of the wall of the cannula end (1).

6. An instrument according to claim 1, characterized by the fact that at least one recess (4) is constructed as a radial deformation of the wall of the cannula end (1).

7. An instrument according to claim 1, characterized by the fact that at least one recess (4) is formed by means of a container set into a breach in the wall of the cannula end (1).

8. An instrument according to claim 1,
characterized by the fact that at least one recess (4) is constructed as a groove running axially in the shell of a cylindrical tube section (1b) of the cannula end (1).

9. An instrument according to claim 3,
characterized by the fact that the knife (2) is constructed as an adenotome, exhibits a distal cutting edge (2a), and is movable axially between a proximal and distal end position, whereby the cutting edge (2a) is located in the proximal end position proximal to the recess (4) and in the distal position distal to the recess (4).

10. An instrument according to claim 9,
characterized by the fact that the distal cannula end (1) between the tip (1a) and the tube section (1b) exhibiting the recess (4) exhibits a radial circular shoulder (1c) at the outer periphery, against which the cutting edge (2a) of the knife (2) stops at the distal end position.

11. An instrument according to claim 3,
characterized by the fact that the knife (2) is movable by means of a proximal operating grip.

12. An instrument according to claim 1,
characterized by the fact that the distal cannula end (1) of the tip (1a) up to and including the tube section (1b) exhibiting the recess (4) is made of a transparent plastic, preferably in one piece.

13. An instrument according to claim 1,
characterized by the fact that the cannula shaft (6) is rigid.

14. An instrument according to claim 1,
characterized by the fact that the cannula shaft (6) is elastically flexible.

15. An instrument according to claim 12, characterized by the fact that the cannula shaft (6) consists of a metal tube and the distal cannula end (1) is fastened to this metal tube.

16. An instrument according to claim 12, characterized by the fact that the cannula shaft (6) consists of a flexible plastic tube and the distal cannula end (1) is fastened to this plastic tube or is constructed as a piece integral with this plastic tube.

17. An instrument according to one claim 1,
characterized by the fact that the cannula end (1) or preferably the knife (2) exhibits on the outer surface at least one coagulation device.

18. An instrument according to claim 17,
characterized by the fact that the coagulation device is formed by means of an electrically heatable zone (5) of the outer surface.

19. An instrument according to claim 17,
characterized by the fact that the coagulation device is movable by moving the knife (2) and/or the cannula in the area of the preceding sample excision (4).

20. An instrument according to claim 19,
characterized by the fact that the coagulation device is disposed on the outer surface of the knife (2) so that it acts as a surface cover for the recess (4) if the knife (2) is moved over the recess (4).

21. An instrument according to claim 1,
characterized by the fact that a flushing channel is disposed in the cannula shaft (6).

22. An instrument according to claim 1,
characterized by the fact that a suction channel is disposed in the cannula shaft (6) leading into the recess (4).

23. An instrument according to claim 1,
characterized by the fact that the space between the cannula shaft (6) and the knife (2) is constructed as a flushing channel and/or a suction channel.

24. An instrument according to claim 1,
characterized by the fact that at the distal cannula end (1) a marker ring can be introduced which can be stripped off from the cannula end (1) to behind in the body duct.
